(19) United States
(12) Patent Application Publication
(10) Pub. No.: US 2025/0136929 A1
(43) Pub. Date: May 1, 2025

(54) BIOREACTOR

(71) Applicant: Ivy Farm Technologies Limited, Oxford (GB)

(72) Inventors: Kiruthika LAWTON, Oxford (GB); Robin NYLAND, Oxford (GB); Simon William PARTRIDGE, Oxford (GB)

(21) Appl. No.: 18/837,175

(22) PCT Filed: Feb. 8, 2023
(86) PCT No.: PCT/GB2023/050284

§ 371 Date: Aug. 8, 2024

(30) Foreign Application Priority Data
Feb. 8, 2022 (GB) 2201609.1

(51) Int. Cl. C12N 5/00; C12M 1/00

(52) U.S. Cl. CPC C12N 5/0068; C12M 29/10; C12N 2533/40; C12N 2533/74; C12N 2539/00

(57) ABSTRACT

There is provided a method of culturing cells, wherein the method comprises: introducing seed cells onto a first surface of a first substrate; culturing cells on the first surface of the first substrate within a bioreactor, wherein culture medium is introduced over a second surface of the first substrate and permeates through the substrate to the first surface; wherein the second surface is on the opposite side of the substrate to the first surface. Also provided is a bioreactor system comprising a bioreactor; a first substrate within the bioreactor, wherein the first substrate has a first surface and a second surface, wherein the second surface is on the opposite side of the first substrate to the first surface, and the first surface is in fluid communication with the second surface; wherein the bioreactor is configured to introduce cell culture medium to the second surface of the first substrate.

### FIELD

The present invention relates to a method of culturing cells. The present invention further relates to a substrate assembly for culturing cells. The present invention also relates to a bioreactor system.

### BACKGROUND OF THE INVENTION

There are concerns about the sustainability of traditional farming practices relating to the rearing of livestock. These traditional farming practices are energy intensive, land intensive and utilise a large amount of antibiotics. A possible approach for addressing these concerns is culturing of meat in a bioreactor. This requires far less energy and land and is a desirable approach for feeding the world's growing population.

However, the success of meat culture depends on the commercial viability of the culture process. The present invention provides means for increasing meat culture viability and efficiency.

### SUMMARY OF THE INVENTION

The present invention relates to a method of culturing cells, wherein the method comprises the following steps: introducing seed cells onto a first surface of a first substrate; culturing cells on the first surface of the first substrate within a bioreactor, wherein culture medium is introduced over a second surface of the first substrate and permeates through the substrate to the first surface; and wherein the second surface is on the opposite side of the substrate to the first surface.

The present invention allows the separation of the surface on which the cells are grown from the surface on which the cell culture medium is introduced. This allows the cells to grow such that they can be undisturbed by the introduction of the cell culture medium. This can allow high flow rates for replenishing the cell culture medium to be used, while also avoiding the high shear stress caused by high flow rates damaging or dislodging the cells. It is preferred that the flow rate of fluid over the first surface is less than the flow rate of fluid over the second surface during the cell culturing process, in particular that the peak flow rate over the first surface is less than the peak flow rate over the second surface during the cell culturing process. It is particularly preferred that there is substantially no fluid flow above the first surface of the substrate during the cell culturing process. In other words, there are stagnant conditions above the first surface.

The present invention can be generally used for the culture of cells, in particular adherent cells (cells that grow on a substrate, also known as anchorage-dependent cells). The invention is particularly advantageous for culturing muscle and/or fat cells for the production of meat for consumption, e.g. human consumption. Thus, the cells introduced (seeded) onto the substrate may be capable of differentiating into myocytes (including myotubes) and/or adipocytes. In particular, the cells introduced may be stem cells, progenitor cells, or precursor cells. Exemplary cells include embryonic stem cells (ESCs), induced pluripotent stem cells (iPSCs), mesenchymal stem cells (MSCs), muscle stem cells (muscle satellite cells), myoblasts, pre-adipocytes, or combinations thereof. Myoblasts are particularly preferred. The cells introduced may be primary cells or cell lines. Exemplary cell lines include Chinese Hamster Ovary (CHO) cells and C2C12 cells (a myoblast subclone).

During cell culture in the bioreactor, the cells or a portion of the cells may differentiate into myocytes (including myotubes) and/or adipocytes (including white fat cells and brown fat cells). Myocytes (including myotubes) are particularly preferred. Thus, the invention may be used for differentiating cells.

### DETAILED DESCRIPTION OF THE INVENTION

Cells for use in the invention may be of any animal origin. However, typically, the cells are not human cells. In particular, preferably, the cells are not produced through the destruction of human embryos. The cells are typically cells of non-human animals, such as non-human mammals, birds, fish, crustaceans, molluscs, reptiles, amphibians, or insects. Exemplary non-human mammals include those in the genera Bovinae, Camelidae, Canidae, Caprae, Cervidae, Felidae, Equidae, Lagomorphs, Macropodidae, Oves, Rodents, or Suidae. The cells may be cells of livestock or poultry. The cells may be porcine, bovine, ovine, caprine, avine, or piscine.

Seed cells are first introduced on to a first surface of a first substrate. Seed cells refers to the cells that are initially introduced and which will proliferate and/or differentiate while in the bioreactor over the course of the cell culturing.

The bioreactor that can be used with the present invention is any suitable bioreactor for culturing adherent cells. In general terms, a bioreactor is a vessel that can support the maintenance and growth of cells. This is also referred to as the culture of cells.

The bioreactor houses the substrate along with cell culture medium, and optionally cells. The cell culture medium may contain nutrients and growth factors within a fluid that supports the maintenance, proliferation, and growth of the cells. The cell culture medium may contain factors promoting cell differentiation. An exemplary culture medium is Dulbecco's Modified Eagle Medium (DMEM). Cell culture medium for use in the invention may comprise serum or may not comprise serum. The cells that are adhered on the substrate are submerged in the cell culture medium. The cell culture medium may be moved around the bioreactor to facilitate the growth of the cells.

A particularly preferred approach is to use perfusion of the cell culture medium. Perfusion refers to the introduction of medium into the bioreactor while removing medium (e.g. comprising lactic acid) from the bioreactor. It is particularly preferred that the new medium is introduced at the bottom of the bioreactor and the spent medium is removed from the top of the bioreactor. The new medium can be introduced by being pumped into the bioreactor. The spent medium may be removed by being pumped out of the bioreactor. The introduced medium may be fresh medium, in the sense it has not been previously introduced into the bioreactor and the removed medium can be spent medium, in the sense that it has been exposed to growing cells. However, the perfusion approach encompasses recirculating, at least part of, the medium through the bioreactor.

The bioreactor can regulate the temperature of the medium within which the cells are growing. This allows optimization of the cell growth.

The bioreactor may be any suitable size for culturing the cells. Accordingly, the bioreactor may have a capacity of at least 1 litre, or at least 2 litres, or at least 3 litres, or at least 5 litres, or at least 10 litres, or at least 20 litres, or at least 50 litres. A bioreactor system may be employed that contains a plurality of bioreactors, where each bioreactor is used to culture cells on its own substrate.

The substrate can be any suitable form that has a first surface and a second surface, where the first surface is on the opposite side of the substrate to the second surface. The presence of the first surface and second surface on opposite sides of the substrate means that you can travel from the first surface to the second surface through the body of the substrate. It is particularly preferred that the first surface is substantially parallel to the second surface. This allows a constant distance between the first surface and second surface, and so uniform performance across the substrate.

The substrate can be in a substantially planar form, i.e. both the first surface and the second surface are of a flat and level form. This is particularly useful approach for combining multiple substrates together, where they can each be conveniently stacked together as described herein. It also allows for a large area of the substrate to be exposed to the optimum cell culturing conditions. It also allows fluid, e.g. cell culture media, to flow easily across the substrate.

The substrate allows fluid to permeate through it. In other words, the first surface of the substrate is in fluid communication with the second surface of the substrate. This fluid communication may be achieved in any suitable manner. For example, the substrate may have a suitable degree of open porosity. However, in general, the pores should not allow passage of cells. A particularly useful approach to achieve the required fluid communication is to form the substrate from a plurality of fibres. In this way, there can be the required porosity between the fibres.

The substrate may comprise a plurality of fibres. In particular, the substrate may consist essentially of a plurality of fibres, or consist of a plurality of fibres. The fibres may be arranged so as to have a random orientation, i.e., the fibres may be arranged so as to have no substantial alignment. A substantial alignment may be an alignment where a majority of the total fibre length present runs along a direction that is within 30°, preferably within 20°, or more preferably within 10° of a certain direction. Further, a substantial alignment may be an alignment where the majority of the total fibre length present runs along a certain direction. The direction along which the fibre runs is the tangential direction at each position as you move along the fibre.

Alternatively, the fibres are arranged to be substantially aligned. As noted above, the fibres may be considered to be substantially aligned when a majority of the total fibre length present runs along a direction that is within 10° of a certain direction. In particular, the alignment may be a substantial alignment where the majority of the total fibre length present runs along a certain direction. As used herein, the majority refers to greater than 50% of the fibre length relative to the total fibre length. However, when the term majority is used, it is particularly preferred that this feature applies to at least 60% of the fibre length relative to the total fibre length, even more preferably at least 70% of the fibre length relative to the total fibre length, even more preferably at least 80% of the fibre length relative to the total fibre length, or at least 90% or at least 95%. It is particularly preferred that the majority refers to all of the total fibre length that is present.

The substrate may comprise a region where the fibres are substantially aligned and a region where the fibres have a random orientation. The random orientation is able to provide beneficial structural properties due its overall isotropic nature, while the substantially aligned region can be utilised to advantageously influence the growth of the cells, e.g. myotubes. Accordingly, it is particularly preferred that the fibres at the first surface are substantially aligned and the fibres at the second surface are randomly oriented. In particular, the bulk (the majority of the fibres relative to the total fibre length) may be randomly oriented to provide the structural support, as long as at least the fibres of the first surface are substantially aligned.

It is preferred that all of the fibres are formed of the same material. This reduces the complexity associated with cultivating meat on the substrate, since only one type of substrate material has to be accounted for in any subsequent processing steps. The cultivated cells may be removed from the substrate for further processing.

Once removed from the substrate, the cells may be used directly as the meat product for consumption. Alternatively, the cells may be biomass which is further processed into a meat product for consumption. Thus, the methods of the invention may further comprise a step of processing the cells into a meat product for consumption, e.g. for human consumption.

The fibres may be formed from polycaprolactone, poly L lactic acid (PLLA), or poly L glycolic acid (PLGA).

The fibres may be edible. Edible fibres are those which can be safely eaten by humans and/or are considered safe for eating by humans, in line with the common understanding of edible products. Edible fibres may comprise substantially, consist essentially of, or consist of materials designated Generally Recognized as Safe (GRAS) by the US Food and Drug Administration (FDA), in particular under sections 201(s) and 409 of the Federal Food, Drug, and Cosmetic Act (FDCA) and/or materials which comply with the Food Chemicals Codex (FCC). The edible fibres may be digestible by humans. The edible fibres may have nutritional value for humans. Suitable edible fibres are formed from polysaccharides. A preferred edible fibre is formed from alginate. This edible substrate provides a reduced risk of contamination with non-edible materials when incorporating the resulting cell culture into a final product that is intended to be eaten. In particular, the cultured meat on the substrate can be incorporated at least partially into the final product for consumption along with the edible substrate itself. This vastly simplifies the processing of the product.

The fibres may be manufactured by any suitable method. A particularly preferred approach includes electrospinning of the fibre. The electrospun fibre can be wound onto a drum and the rotational speed of the drum can be adjusted to change the orientation of the fibres. A relatively low speed will lead to the fibre being wound on to the drum with a random orientation, while a relatively high speed will lead to the fibre being wound on to the drum in a substantially aligned manner. By adjusting the speed of the drum during the electrospinning process, it is possible to adjust the alignment of the wound fibre. In particular, it is preferred to rotate the drum at a relatively low speed for the majority of the electrospinning process and use a relatively high speed at either the beginning of the process or the end of the process so as to have aligned fibres on one of the spun surfaces. The collection of spun fibres on the drum can be cut in the longitudinal direction of the drum, removed from the drum and flattened in order to give the final substrate.

The substrate may be processed into a substrate assembly. The substrate assembly may comprise a substrate described herein and a support contacting the substrate. The support can be of any suitable form. The support can be configured so as to provide structural support to the substrate. The support can also be configured so as to assist the placement of the substrate within the bioreactors, for example, it may be configured in a complementary manner to support-receiving portions that are within the bioreactor. In this manner, the support assembly can be readily incorporated into the bioreactor in a reliable way.

When a plurality of substrates are utilised with the present invention, the details that are described herein in relation to the substrate are applicable to all of the substrates of the plurality. For example, when a first substrate and a second substrate are present, the features described herein in relation to the substrate are applicable to both substrates. It is particularly preferred that features that are described herein apply to each of the substrates of the plurality of substrates, for example, each of the substrates may be formed from a plurality of fibres and have a first surface having substantially aligned fibres and a second surface having randomly oriented fibres. Having consistent features for the plurality of substrates results in consistent performance across these substrates when they are utilised together in the bioreactor.

The present invention may utilise a first substrate and a second substrate, wherein both the first substrate and the second substrate each have a first surface and a second surface, wherein the second surface is on the opposite side of the respective substrate to the first surface.

The first and second substrates can be arranged in the bioreactor such that the second surface of the second substrate opposes the second surface of the first substrate so as to bound a perfusion region. The term ‘perfusion region’ is used herein to denote a region that is intended to be the region into which cell culture medium is introduced. In this way, the second surfaces of both substrates are experiencing the same environment and same forces due to fluid flow. The first substrate and second substrate bound the perfusion region in the sense that they form part of the boundary of the region, this does not necessarily mean that the substrates fully enclose the perfusion region. The perfusion region may be fully enclosed by a combination of the substrates and other components of the substrate assemblies (e.g. a support) and/or the bioreactor. In this way, the invention can ensure there is no fluid communication from the perfusion region to the first surface of the substrates apart from through the body of the substrates.

In use, the culture medium may be introduced into the perfusion region so as to introduce the culture medium over the second surface of the first substrate and the second surface of the second substrate so as to permeate through the first substrate to the first surface of the first substrate and permeate through the second substrate to the first surface of the second substrate. In this way, cell culture medium only has to be introduced into the perfusion region for it to reach the first surfaces of both of the substrates.

In addition to the presence of a first substrate and a second substrate, there may also be a third substrate, wherein the third substrate has a first surface and a second surface, wherein the second surface is on the opposite side of the third substrate to the first surface.

The third substrate, when present, may be arranged in the bioreactor such that the first surface of the third substrate opposes the first surface of the second substrate so as to bound a cell region. The use of the term ‘cell region’ herein denotes a region within which it is intended for the cells to be cultured. In this way, the first surfaces are experiencing the same environment which may be optimised for cell growth. The second substrate and third substrate bound the cell region in the sense that they form part of the boundary of the region, this does not necessarily mean that the substrates fully enclose the cell region. The cell region may be fully enclosed by a combination of the substrates and other components of the substrate assemblies (e.g. a support) and/or the bioreactor. In this way, the invention can ensure there is no fluid communication from the cell region to the second surface of the substrates apart from through the body of the substrates.

As noted above, the cell culture medium may be introduced into the perfusion region. When a third substrate is also present, the cell culture medium is additionally introduced over the second surface of the third substrate so that it can permeate through the third substrate to its first surface. In this way, cell culture medium can be provided to the cells that are cultured on the first surface of the third substrate.

Instead of just an additional third substrate, there may be at least one additional pair of substrates. The at least one additional pair of substrates is in addition to the first substrate and second substrate described herein. This pair of substrates may be arranged and configured in the same manner as the first and second substrate. In this way, the additional pair behaves in the same manner as the first substrate and the second substrate but provides more surface upon which to culture cells within the bioreactor. In this sense, a modular system is provided where there can be any number of additional pairs of substrates allowing the process to be scaled-up within one bioreactor. For example, there can be two or more additional pairs of substrates, five or more additional pairs of substrates, or ten or more additional pairs of substrates.

When using this modular approach, the at least one additional pair can be arranged such that it is arranged over the first and second substrate such that the first substrate, the second substrate and the at least one additional pair of substrates forms a stacked arrangement. In other words, the substrates can be arranged in a stack such that the second substrate is positioned above the first substrate, the substrate that is equivalent to the first substrate in the first additional pair of substrates is then positioned over the second substrate, the substrate that is equivalent to the second substrate in the first additional pair of substrates is then positioned over the substrate that is equivalent to the first substrate in the first additional pair, and then any further additional pairs of substrate are then stacked in turn. The use of the term ‘over’ denotes a relative arrangement between the substrates and does not necessitate the stack being a vertical stack.

This stacking arrangement allows further perfusion regions to exist within each of the additional pairs of substrates that is present. Further, there is a cell region between the first additional pair of substrates and the second substrate and between each of any additional pairs of substrates that may be present. In this manner there can be alternating perfusion regions and cell regions along the stack. This arrangement effectively shares the perfusion region between two adjacent substrates and the cell region between two adjacent substrates. This is a particularly effective way of arranging the substrates to minimise the amount of components required to provide cell culture medium to the plurality of substrates.

Accordingly, the present invention provides a bioreactor system comprising the various configurations of substrates described herein. Each of the perfusion regions in the bioreactor system may be configured to introduce cell culture medium and, optionally, to remove the cell culture medium. Accordingly, the flow of cell culture medium can be provided for each perfusion region. The bioreactor system can also be simplified by each of the cell regions not comprising any components that would allow the introduction of cell culture medium into those regions.

Exemplary embodiments of the present invention are depicted in FIG. 1. FIG. 1a depicts a first substrate having a first surface formed from a plurality of substantially aligned fibres and a second surface formed from a plurality of randomly aligned fibres, wherein the second surface is on the opposite side of the substrate to the first surface, and wherein the first surface is in fluid communication with the second surface. FIG. 1b depicts the first substrate of FIG. 1a and a second substrate configured in the same way as the first substrate, wherein the first substrate and the second substrate are arranged such that the second surface of the first substrate opposes the second surface of the second substrate so as to bound a perfusion region. FIG. 1c depicts the first substrate and the second substrate of FIG. 1b and a third substrate configured in the same way as the first and second substrate, wherein the third substrate is arranged such that the first surface of the third substrate opposes the first surface of the second substrate so as to bound a cell region. FIG. 1d depicts the first and second substrate of FIG. 1b and additional pairs of substrates, each of the pairs of substrates being arranged and configured in the same way as the first and second substrate of FIG. 1b, and wherein each pair is arranged such that it is arranged over the first and second substrate such that the first substrate, the second substrate and each additional pair of substrates forms a stacked arrangement comprising further perfusion regions.

A coating may be applied to the substrate, for example the first surface having substantially aligned fibres. The coating may be applied to the substrate before placing the substrate in the bioreactor. Thus, the substrate, for example the first surface having substantially aligned fibres, may have a coating.

The coating may comprise a peptide for enhancing attachment (adherence) of the cells to the substrate. The peptide may be, or may be derived from, an extracellular matrix (ECM) protein, such as laminin. Alternatively, the peptide may be a synthetic peptide.

The substrate may be subjected to a treatment step. For example, the substrate may be subjected to plasma treatment and/or a UV treatment step. This can assist with subsequent cell adhesion to the substrate.

The cultured cells may be used as a replacement for livestock-derived meat in a meat product for consumption using suitable food production techniques as are well known to the skilled person. For example, the cultured cells may be simply added into a mixture intended to form meatballs or sausages as a replacement or addition to livestock-derived meat, and the mixture then processed into the meat product using conventional techniques.

Singular encompasses plural and vice versa. For example, although reference is made herein to “a” particulate substrate, “a” flow distributor, and the like, one or more of each of these and any other components can be used.

The terms “comprising” and “comprises” as used herein are synonymous with “including”, “includes” or “containing”, “contains”, and are inclusive or open-ended and do not exclude additional, non-recited members, elements or method steps.

Additionally, although the present invention has been described in terms of “comprising”, the invention as detailed herein may also be described as “consisting essentially of” or “consisting of”.

Although the present invention has been described in terms of “obtainable by”, the associated features of the present invention detailed herein may also be independently described as “obtained by”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Where ranges are provided in relation to a genus, each range may also apply additionally and independently to any one or more of the listed species of that genus.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

The present invention will now be described with reference to the following Figures and examples.

### FIGURES

FIG. 1 depicts exemplary embodiments of the invention.

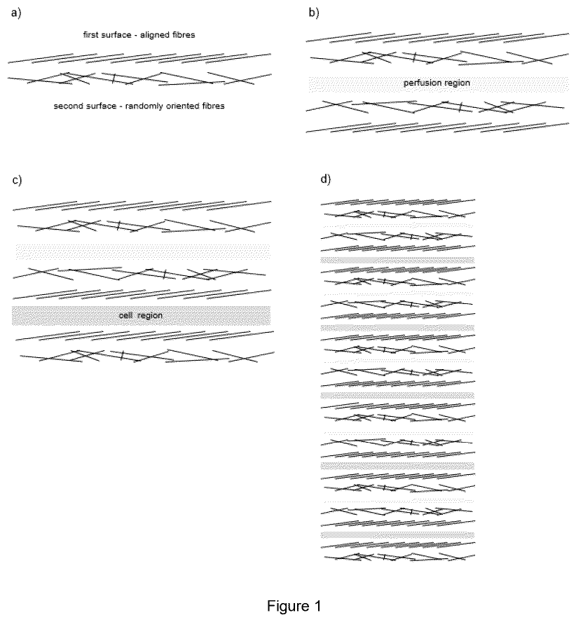

FIG. 2 depicts cells attached to a 2D 12WP (left panel), a layer of electrospun PCL aligned fibres (middle panel), or a layer of randomly aligned fibres (right panel). Blue staining=DAPI stained nuclei; red staining=rhodamine phalloidin stained actin cytoskeleton; green straining=MF20 (myosin heavy chain antibody).

### EXAMPLES

### Example 1. Substrate Production

Polycaprolactone (PCL) fibres are spun using a rotating drum. This electrospinning process utilises a single needle (20 gauge diameter). The solution for spinning the fibres is polycaprolactone, 12 mass %, in chloroform. A flow rate of 0.05 ml/min is used and a voltage of positive 12 kV and negative 2 kV. The drum rotational speed is varied where the random layer of the resulting substrate is spun with the drum rotating at 100 RPM for 30 minutes and then the aligned layer is spun with the drum rotating at 1000 RPM for 30 minutes. The ‘mat’ that is formed on the drum is then cut to remove it so it can be used in a bioreactor as a substrate.

An alternative substrate was produced from alginate. The electrospinning process was conducted using a single needle (18 gauge diameter) with a medium viscosity alginate solution 2 wt % in water. Polyethylene Glycol 600,000 Mw 5 wt % in water was mixed with the alginate solution in a ratio of around 2:1. The flow rate used was 0.01 ml/min and the voltage was positive 18 kV and negative 2 kV. The drum was initially rotated at 100 RPM for 30 minutes to produce the random layer and then for 30 minutes at 1000 RPM to produce the aligned layer.

### Example 2. Myotube Formation on Aligned Fibres

Substrates comprising a layer of electrospun PCL aligned fibres or randomly oriented fibres were prepared. The PCL was treated with plasma to increase wettability and cell adhesion, sterilised with UV light, and washed three times with PBS. The substrates were then transferred into 12 well plates (12WP) and coated with laminin to enhance cell adhesion. Primary porcine myoblasts were seeded onto the substrates, or the 2D surface of a 12WP as a control, and cultured in DMEM at 37 C.

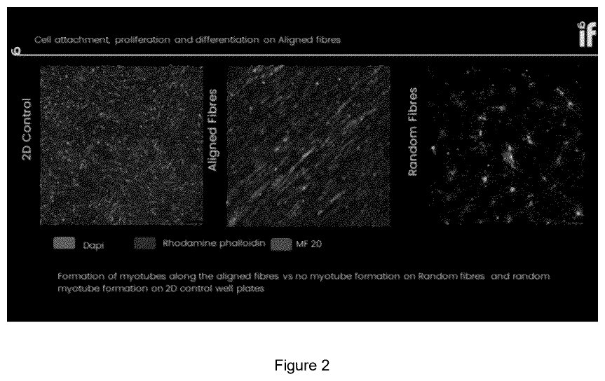

After 7 days of culture, the cells were stained with DAPI to observe the nuclei, rhodamine phalloidin to observe the actin cytoskeleton, and MF20 to observe myotube formation. As shown in FIG. 2, myotubes formed along the aligned fibres (middle panel), but did not form on the randomly oriented fibres (right panel) nor the 2D 12WP control (left panel).

1. A method of culturing cells, wherein the method comprises the following steps:
introducing seed cells onto a first surface of a first substrate;
culturing cells on the first surface of the first substrate within a bioreactor, wherein culture medium is introduced over a second surface of the first substrate and permeates through the substrate to the first surface; and
wherein the second surface is on the opposite side of the substrate to the first surface.

2. The method of claim 1 further comprising a second substrate, wherein the second substrate has a first surface and a second surface, wherein the second surface is on the opposite side of the second substrate to the first surface, the second substrate being arranged in the bioreactor such that the second surface of the second substrate opposes the second surface of the first substrate so as to bound a perfusion region, wherein the culture medium is introduced into the perfusion region so as to introduce the culture medium over the second surface of the first substrate and the second surface of the second substrate so as to permeate through the first substrate to the first surface of the first substrate and permeate through the second substrate to the first surface of the second substrate.

3. The method of claim 2, further comprising a third substrate, wherein the third substrate has a first surface and a second surface, wherein the second surface is on the opposite side of the third substrate to the first surface, the third substrate being arranged in the bioreactor such that the first surface of the third substrate opposes the first surface of the second substrate so as to bound a cell region, wherein the culture medium is additionally introduced over the second surface of the third substrate and permeates through the third substrate to its first surface.

4. The method of claim 2, further comprising at least one additional pair of substrates, the pair of substrates being arranged and configured in the same manner as the first and second substrate, and wherein the at least one additional pair is arranged such that it is arranged over the first and second substrate such that the first substrate, the second substrate and the at least one additional pair of substrates forms a stacked arrangement comprising further perfusion regions within each of the at least one additional pair of substrates and cell regions between the at least one additional pair of substrates and the second substrate and between any further additional pairs of substrates.

5. A substrate assembly for culturing cells, comprising
a first substrate formed from a plurality of fibres, wherein the first substrate has a first surface and a second surface, wherein the second surface is on the opposite side of the substrate to the first surface; and
a support contacting the first substrate; and
wherein the first surface has substantially aligned fibres and the second surface has randomly oriented fibres.

6. A bioreactor system comprising
a bioreactor;
a first substrate within the bioreactor, wherein the first substrate has a first surface and a second surface, wherein the second surface is on the opposite side of the first substrate to the first surface, and the first surface is in fluid communication with the second surface; and
wherein the bioreactor is configured to introduce cell culture medium to the second surface of the first substrate.

7. The bioreactor system of claim 6 further comprising:
a second substrate within the bioreactor, wherein the second substrate has a first surface and a second surface, wherein the second surface is on the opposite side of the second substrate to the first surface, and the first surface is in fluid communication with the second surface; and
wherein the first substrate and the second substrate are arranged such that the second surface of the first substrate opposes the second surface of the second substrate so as to bound a perfusion region of the bioreactor; and
wherein the bioreactor is configured so as to introduce cell culture medium into the perfusion region so as to introduce cell culture medium to the second surface of the first substrate and the second surface of the second substrate.

8. The bioreactor system of claim 7 further comprising:
a third substrate, wherein the third substrate has a first surface and a second surface, wherein the second surface is on the opposite side of the third substrate to the first surface and the first surface is in fluid communication with the second surface, the third substrate being arranged in the bioreactor such that the first surface of the third substrate opposes the first surface of the second substrate so as to bound a cell region, wherein the bioreactor is configured to additionally introduce cell culture medium over the second surface of the third substrate.

9. The bioreactor system of claim 7, further comprising:
at least one additional pair of substrates, the pair of substrates being arranged and configured in the same manner as the first and second substrate, and wherein the pair is arranged such that it is arranged over the first and second substrate such that the first substrate, the second substrate and the at least one additional pair of substrates forms a stacked arrangement comprising further perfusion regions within each of the at least one additional pair of substrates and cell regions between the at least one additional pair of substrates and the second substrate and between any further additional pairs of substrates; and
wherein the bioreactor is further configured so as to introduce cell culture medium into each of the perfusion regions so as to introduce cell culture medium to the second surface of each of the substrates.

10. The method of claim 1, wherein the first substrate, second substrate when present, third substrate when present, and the at least one additional pair of substrates when present, are formed from a plurality of fibres.

11. The method of claim 10, wherein the first substrate, second substrate when present, third substrate when present, and the at least one additional pair of substrates when present, each have aligned fibres on the first surface and randomly oriented fibres on the second surface.

12. The method of claim 10, wherein the plurality of fibres are electrospun fibres.

13. The method of claim 10, wherein the plurality of fibres are edible fibres.

14. The method of claim 10, wherein the first substrate, second substrate when present, third substrate when present, and the at least one additional pair of substrates when present, are each planar substrates.

15. (canceled)
